(19) United States
(12) Patent Application Publication
(10) Pub. No.: US 2025/0032411 A1
(43) Pub. Date: Jan. 30, 2025

(54) A SELF-EMULSIFYING DRUG DELIVERY SYSTEM (SEDDS) OF PUNICIC ACID COMBINED WITH NEUROPROTECTIVE NATURAL PRODUCTS TO DELAY ONSET, PREVENT, OR REVERSE NEURODEGENERATIVE DISEASE

(71) Applicant: TAHIRO INC., Encino, CA (US)

(72) Inventors: Oded SHOSEYOV, Shoham (IL); Avi PALATNIK, Tel Aviv (IL)

(73) Assignee: TAHIRO INC., Encino, CA (US)

(21) Appl. No.: 18/691,330

(22) PCT Filed: Sep. 12, 2022
(86) PCT No.: PCT/IL2022/050988

§ 371 Date: Mar. 12, 2024

Related U.S. Application Data
(60) Provisional application No. 63/243,153, filed on Sep. 12, 2021.

(51) Int. Cl. A61K 9/107; A61K 31/00; A61K 31/202; A61K 36/42; A61K 36/54; A61K 47/10; A61K 47/26; A61P 25/16

(52) U.S. Cl. CPC A61K 9/1075; A61K 31/202; A61K 31/658; A61K 36/3482; A61K 36/42; A61K 36/54; A61K 47/10; A61K 47/26; A61P 25/16

(57) ABSTRACT

This invention is directed to a Self-Emulsifying Drug Delivery System (SEDDS) of Punicic Acid combined with neuroprotective natural products to treat, prevent, delay onset, prevent or reverse neurodegenerative disease.

### RELATED APPLICATION

This application claims the benefit of U.S. Provisional Application No. 63/243,153, filed on Sep. 12, 2021. The entire teachings of the above application(s) are incorporated herein by reference.

### FIELD OF THE INVENTION

This invention is directed to a Self-Emulsifying Drug Delivery System (SEDDS) of Punicic Acid combined with neuroprotective natural products to treat, prevent, delay onset, prevent or reverse neurodegenerative disease.

### BACKGROUND OF THE INVENTION

Punicic acid is the main component of Pomegranate and Snake Gourd oil.

Punicic acid (also called trichosanic acid) is a polyunsaturated fatty acid, 18:3 cis-9, trans-11, cis-13. It is named for the pomegranate, (Punica granatum), and is obtained from pomegranate seed oil. It is also found in the seed oils of snake gourd at levels reaching 40%. Punicic acid has the following formula:

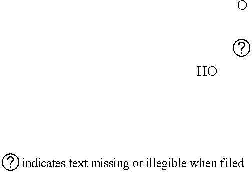 [SMILES: O ; O]

Binyamin et al. (Neurobiology of Disease 108 (2017) 140-147) showed that long term administration of nano pomegranate seed oil (Nano-PSO) is safe and effective in the prevention and/or delay of onset of neurodegenerative conditions such as genetic Creutzfeldt-Jakob Disease (CJD).

### Mannitol

Mannitol was shown to interfere with the aggregation process of alfa-synuclein in vitro (the main cause of Parkinson's disease) and in vivo, in addition to its blood-brain barrier-disrupting properties. See Shaltiel-Karyo (The Journal of Biological Chemistry Vol. 288, No. 24, Pp. 17579-17588, 2013).

When administered to a Drosophila model of Parkinson's Disease, mannitol dramatically corrected behavioral defects and reduced the amount of alpha-synuclein aggregates in the brains of treated flies.

In the mThyl-human alpha-synuclein transgenic mouse model (a Progressive Mouse Model of Parkinson's Disease), a decrease in alpha-synuclein accumulation was detected in several brain regions following treatment, suggesting that mannitol promotes alpha-synuclein clearance in the cell bodies. It appears that mannitol has a general neuroprotective effect in the transgenic treated mice, which includes the dopaminergic system. It was therefore suggested that mannitol acts in a dual therapeutic mechanism in the treatment of Parkinson's disease. One concern is that the dose used in this study was 1 g/kg/day administered interperitoneally to mice and for fly feeding Mannitol was added to standard molasses medium at a concentration of 75 mM (13.65 gr/liter).

A phase II clinical study (Front. Neurol., 3 Jan. 2022 https://doi.org/10.3389/fneur.2021.716126) demonstrated tolerability of oral mannitol in Parkinson's disease, which was assessed by the maximal daily dose (in grams, up to 18 gr for 36 weeks) of mannitol that does not cause discomfort. The study concluded that long-term use of 18 g per day of oral mannitol is safe in Parkinson's disease patients but only two thirds of patients tolerate this maximal dose.

### Cinnamon

As stated by the Alzheimer's Society though some of the extracts of cinnamon may warrant investigation to try and establish new treatments, cinnamon itself is not a treatment for Alzheimer's disease. The levels of cinnamon a person would have to eat to replicate the results of many of the experiments that have taken place would actually be toxic. More research is needed into these chemicals but, if beneficial, they will need to be provided in a drug form rather than in cinnamon. Naturally, the use of the Self-Emulsifying Drug Delivery System (SEDDS) may reduce the effective oral dose.

### Cannabis

As stated by the Alzheimer's Society, currently there is no clinical evidence to show that cannabis or cannabis oil (CBD oil) can stop, reverse or prevent dementia (https://www.alzheimers.org.uk/about-dementia/treatments/alternative-therapies/cannabis-cbd-oil-and-dementia). However, some studies suggest cannabis could help manage a few behavioral symptoms of dementia, such as agitation and aggression.

Δ9-tetrahydrocannabinol (THC) has demonstrated a positive in vivo effect in mice. A chronic low dose of THC restores cognitive function in old mice (see Bilkei-Gorzo, Nature Medicine 2017 Vol 23 Number 6). THC treatment restored hippocampal gene transcription patterns such that the expression profiles of THC-treated mice aged 12 months closely resembled those of THC-free animals aged 2 months. The dose was 3 mg per kg bodyweight per day. This represents 180 mg/60 kg subject-an amount that could create a psychoactive effect. Thus, oil comprising less than 0.3 percent of THC to comply with regulations in many countries will result in consumption of at least 9 ml/60 kg subject. Again, if this effective dose can be reduced to lower values using Punicic acid-SEDDS as a carrier, it would be a practical treatment and prevent psychoactive effects.

### Hericium erinaceus, Lion's mane mushroom

Studies have found that lion's mane mushrooms contain two special compounds that can stimulate the growth of brain cells: hericenones and erinacines. In addition, it was shown to contain polysaccharides with anti-inflammatory effect.

It was shown recently in a double blind placebo controlled clinical trial that oral administration of Hericium erinaceus mycelia capsules (350 mg/capsule; comprising 5 mg/g erinacine A active ingredient) per day for the treatment of patients with mild Alzheimer's Disease (AD) is safe, well-tolerated, and offers neurocognitive benefits (https://www.frontiersin.org/articles/10.3389/fnagi.2020.00155/full).

Pomegranate oil was shown to have neuroprotective activity in animal studies when administered via a Self-Emulsifying Delivery System (SEDDS) thus proving to be effective in bypassing the blood brain barrier.

Other natural compounds such as cannabis oil, CBD, THC, cinnamon extracts, Hericium erinaceus extracts and mannitol have shown neuroprotective activities, however relatively high doses are essential to achieve a significant effect thus negating their usefulness.

Effective formulation selection may be conducted in a relatively short term in vivo Parkinson Drosophila model and consequently on appropriate neuroprotective larger animal (mouse) cognition in vivo model. This invention addresses the need to deliver effective doses of neuroprotective formulations while administering low non-toxic levels of bioactive molecules.

The relatively large levels of CBD, THC, Cinnamon extract, or mannitol which need to be administered via oral administration to treat neurodegenerative diseases means that a new treatment that allows for lower levels of administration of these agents is necessary.

### SUMMARY OF THE INVENTION

The invention is directed to the utility of punicic acid-SEDDS to deliver effective and relatively low levels of CBD, THC, Cinnamon extract (all oil soluble compounds) alone or a mixture thereof, with or without mannitol (a water soluble compound) via oral administration to treat neurodegenerative diseases.

In some embodiments, the present disclosure provides a pharmaceutical composition comprising an oral formulation for the treatment of a neurodegenerative disease comprising at least 20% Punicic acid-SEDDS comprising full spectrum cannabis oil, CBD, THC, Cinnamon oleoresin alone or mixture thereof with or without mannitol.

In some embodiments, the present disclosure provides a pharmaceutical composition comprising an oral formulation for the treatment of a neurodegenerative disease comprising at least 20% Punicic acid-SEDDS comprising full spectrum cannabis oil, CBD, THC, Cinnamon oleoresin alone or mixture thereof with or without mannitol; wherein the ratio between the CBD, THC and Cinnamon oleoresin to the pomegranate or snake gourd oil is between 1:250-1:1 and the total amount of mannitol is between 250 mg up to 20 gr per day.

In some embodiments, the present disclosure provides a method for the treatment of a neurodegenerative disease said method comprising administering an effective amount of an oral formulation comprising at least 20% Punicic acid-SEDDS comprising full spectrum cannabis oil, CBD, THC, Cinnamon oleoresin alone or mixture thereof with or without mannitol.

In some embodiments, the present disclosure provides a method for the treatment of a neurodegenerative disease said method comprising administering an effective amount of an oral formulation comprising at least 20% Punicic acid-SEDDS comprising full spectrum cannabis oil, CBD, THC, Cinnamon oleoresin alone or mixture thereof with or without mannitol; wherein the ratio between the CBD, THC and Cinnamon oleoresin to the pomegranate or snake gourd oil is between 1:250-1:1 and the total amount of mannitol is between 250 mg to 20 gr per day to a subject in need thereof.

In some embodiments, the present disclosure provides a method for preparing an oral formulation for the treatment of a neurodegenerative disease comprising at least 20% Punicic acid-SEDDS comprising full spectrum cannabis oil, CBD, THC, Cinnamon oleoresin alone or mixture thereof with or without mannitol, comprising the following steps:

Mixing 250 mg of an oil comprising at least 20% punicic acid, 350 mg of Tween 80, 155 mg of Span 80 and 50 μl of ethanol using a magnetic stirrer for 20 minutes.

Adding 10 μl of the mixture obtained in the preceding step to 3 ml of deionized water and vortexing for 30-60 seconds.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1A shows the effect of cannabis extracts and cinnamon extracts combined with Pomegranate or snake-gourd to cannabis at different ratios on SHSY-5Y neurons. The Y axis illustrates the neuroprotective effect of the different combinations as compared to control.

FIG. 1B shows the effect of cannabis extracts and cinnamon extracts combined in a ratio of Pomegranate or snake-gourd to cannabis or cinnamon 12:1 on SHSY-5Y neurons. The Y axis illustrates the neuroprotective effect of the different combinations as compared to control.

FIG. 1C shows Pomegranate or snake-gourd to cannabis or cinnamon at 12:1 ratios in different combination screens without mannitol. The Y axis illustrates the neuroprotective effect of the different combinations as compared to control.

FIG. 2 shows the results of the Y-Maze test in vivo mice cognitive study. The graph shows a neutral control, a negative MK-801 control, and a combination of Pomegranate and CBD. The Y axis shows % of entries in the novel arm of the maze.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In some embodiments, provided herein is a pharmaceutical composition comprising Punicic acid-SEDDS comprising any one of full spectrum cannabis oil, CBD, THC, Cinnamon oleoresin, or any combination thereof with or without mannitol.

In some embodiments, provided herein is a pharmaceutical composition comprising at least 20% Punicic acid-SEDDS comprising any one of full spectrum cannabis oil, CBD, THC, Cinnamon oleoresin, or any combination thereof.

In some embodiments, provided herein is a pharmaceutical composition comprising at least 20% Punicic acid-SEDDS and full spectrum cannabis oil.

In some embodiments, provided herein is a pharmaceutical composition comprising at least 20% Punicic acid-SEDDS and CBD.

In some embodiments, provided herein is a pharmaceutical composition comprising at least 20% Punicic acid-SEDDS, THC and Cinnamon oleoresin.

In some embodiments, provided herein is a pharmaceutical composition comprising at least 20% Punicic acid-SEDDS and Cinnamon oleoresin.

In some embodiments, provided herein is a pharmaceutical composition comprising at least 20% Punicic acid-SEDDS, full spectrum cannabis oil, and CBD.

In some embodiments, provided herein is a pharmaceutical composition comprising at least 20% Punicic acid-SEDDS, cannabis oil, and THC.

In some embodiments, provided herein is a pharmaceutical composition comprising at least 20% Punicic acid-SEDDS, cannabis oil, CBD, and Cinnamon oleoresin.

In some embodiments, provided herein is a pharmaceutical composition comprising at least 20% Punicic acid-SEDDS, cannabis oil, THC, and Cinnamon oleoresin.

In some embodiments, provided herein is a pharmaceutical composition comprising at least 20% Punicic acid-SEDDS, CBD, THC, and Cinnamon oleoresin.

In some embodiments, provided herein is a pharmaceutical composition at least 20% Punicic acid-SEDDS, CBD, and THC.

In some embodiments, provided herein is a pharmaceutical composition comprising at least 20% Punicic acid-SEDDS, CBD and Cinnamon oleoresin.

In some embodiments, provided herein is a pharmaceutical composition comprising at least 20% Punicic acid-SEDDS, THC, and Cinnamon oleoresin.

In some embodiments, the pharmaceutical composition comprises at least 25% Punicic acid-SEDDS comprising any one of full spectrum cannabis oil, CBD, THC, Cinnamon oleoresin, or any combination thereof.

In some embodiments, the pharmaceutical composition comprises at least 30% Punicic acid-SEDDS comprising any one of full spectrum cannabis oil,

CBD, THC, Cinnamon oleoresin, or any combination thereof.

In some embodiments, the pharmaceutical composition comprises at least 35% Punicic acid-SEDDS comprising any one of full spectrum cannabis oil, CBD, THC, Cinnamon oleoresin, or any combination thereof.

In some embodiments, the pharmaceutical composition comprises at least 40% Punicic acid-SEDDS comprising any one of full spectrum cannabis oil, CBD, THC, Cinnamon oleoresin, or any combination thereof.

In some embodiments, the pharmaceutical composition comprises at least 20% Punicic acid-SEDDS comprising full spectrum cannabis oil, CBD, THC, Cinnamon oleoresin alone or mixture thereof and mannitol.

In some embodiments, provided herein is a pharmaceutical composition comprising at least 20% Punicic acid-SEDDS, full spectrum cannabis oil, and mannitol.

In some embodiments, provided herein is a pharmaceutical composition comprising at least 20% Punicic acid-SEDDS, CBD, and mannitol.

In some embodiments, provided herein is a pharmaceutical composition comprising at least 20% Punicic acid-SEDDS, THC, Cinnamon oleoresin, and mannitol.

In some embodiments, provided herein is a pharmaceutical composition comprising at least 20% Punicic acid-SEDDS, Cinnamon oleoresin, and mannitol.

In some embodiments, provided herein is a pharmaceutical composition comprising at least 20% Punicic acid-SEDDS, full spectrum cannabis oil, CBD, and mannitol.

In some embodiments, provided herein is a pharmaceutical composition comprising at least 20% Punicic acid-SEDDS, cannabis oil, THC, and mannitol.

In some embodiments, provided herein is a pharmaceutical composition comprising at least 20% Punicic acid-SEDDS, cannabis oil, CBD, Cinnamon oleoresin, and mannitol.

In some embodiments, provided herein is a pharmaceutical composition comprising at least 20% Punicic acid-SEDDS, cannabis oil, THC, Cinnamon oleoresin, and mannitol.

In some embodiments, provided herein is a pharmaceutical composition comprising at least 20% Punicic acid-SEDDS, CBD, THC, Cinnamon oleoresin, and mannitol.

In some embodiments, provided herein is a pharmaceutical composition at least 20% Punicic acid-SEDDS, CBD, THC, and mannitol.

In some embodiments, provided herein is a pharmaceutical composition comprising at least 20% Punicic acid-SEDDS, CBD, Cinnamon oleoresin, and mannitol.

In some embodiments, provided herein is a pharmaceutical composition comprising at least 20% Punicic acid-SEDDS, THC, Cinnamon oleoresin, and mannitol.

In some embodiments, the pharmaceutical composition comprises Punicic at least 25% acid-SEDDS comprising full spectrum cannabis oil, CBD, THC, Cinnamon oleoresin alone or mixture thereof and mannitol.

In some embodiments, the pharmaceutical composition comprises at least 30% Punicic acid-SEDDS comprising full spectrum cannabis oil, CBD, THC, Cinnamon oleoresin alone or mixture thereof and mannitol.

In some embodiments, the pharmaceutical composition comprises at least 35% Punicic acid-SEDDS comprising full spectrum cannabis oil, CBD, THC, Cinnamon oleoresin alone or mixture thereof and mannitol.

In some embodiments, the pharmaceutical composition comprises at least 40% Punicic acid-SEDDS comprising full spectrum cannabis oil, CBD, THC, Cinnamon oleoresin alone or mixture thereof and mannitol.

In some embodiments, the present disclosure provides a method for preparing an oral formulation for the treatment of a neurodegenerative disease comprising at least 20% Punicic acid-SEDDS comprising full spectrum cannabis oil, CBD, THC, Cinnamon oleoresin alone or mixture thereof with or without mannitol, comprising the following steps:

Mixing 250 mg of an oil comprising at least 20% punicic acid, 350 mg of Tween 80, 155 mg of Span 80 and 50 μl of ethanol using a magnetic stirrer for 20 minutes.

Adding 10 μl of the mixture the mixture obtained in the preceding step to 3ml of deionized water and vortexing for 30-60 seconds.

In some embodiments, provided herein is a pharmaceutical composition comprising an oral formulation for the treatment of a neurodegenerative disease.

In some embodiments, provided herein is a pharmaceutical composition comprising an oral formulation for the treatment of a neurodegenerative disease. comprising at least 20% Punicic acid-SEDDS comprising full spectrum cannabis oil, CBD, THC, Cinnamon oleoresin alone or mixture thereof with or without mannitol.

In some embodiments, provided herein is a pharmaceutical composition comprising an oral formulation for the treatment of a neurodegenerative disease. comprising at least 25% Punicic acid-SEDDS comprising full spectrum cannabis oil, CBD, THC, Cinnamon oleoresin alone or mixture thereof with or without mannitol.

In some embodiments, provided herein is a pharmaceutical composition comprising an oral formulation for the treatment of a neurodegenerative disease. comprising at least 30% Punicic acid-SEDDS comprising full spectrum cannabis oil, CBD, THC, Cinnamon oleoresin alone or mixture thereof with or without mannitol.

In some embodiments, provided herein is a pharmaceutical composition comprising an oral formulation for the treatment of a neurodegenerative disease. comprising at least 35% Punicic acid-SEDDS comprising full spectrum cannabis oil, CBD, THC, Cinnamon oleoresin alone or mixture thereof with or without mannitol.

In some embodiments, provided herein is a pharmaceutical composition comprising an oral formulation for the treatment of a neurodegenerative disease. comprising at least 40% Punicic acid-SEDDS comprising full spectrum cannabis oil, CBD, THC, Cinnamon oleoresin alone or mixture thereof with or without mannitol.

In some embodiments, provided herein is a pharmaceutical composition comprising an oral formulation for the treatment of a neurodegenerative disease. comprising at least 20% Punicic acid-SEDDS comprising full spectrum cannabis oil, CBD, THC, Cinnamon oleoresin alone or mixture thereof with or without mannitol; wherein the ratio between the CBD, THC and Cinnamon oleoresin to the pomegranate or snake gourd oil is between 1:250-1:1 and the total amount of mannitol is between 250 mg to 20 gr per day.

In some embodiments, provided herein is a pharmaceutical composition comprising an oral formulation for the treatment of a neurodegenerative disease. comprising at least 25% Punicic acid-SEDDS comprising full spectrum cannabis oil, CBD, THC, Cinnamon oleoresin alone or mixture thereof with or without mannitol; wherein the ratio between the CBD, THC and Cinnamon oleoresin to the pomegranate or snake gourd oil is between 1:250-1:1 and the total amount of mannitol is between 250 mg to 20 gr per day.

In some embodiments, provided herein is a pharmaceutical composition comprising an oral formulation for the treatment of a neurodegenerative disease. comprising at least 30% Punicic acid-SEDDS comprising full spectrum cannabis oil, CBD, THC, Cinnamon oleoresin alone or mixture thereof with or without mannitol; wherein the ratio between the CBD, THC and Cinnamon oleoresin to the pomegranate or snake gourd oil is between 1:250-1:1 and the total amount of mannitol is between 250 mg to 20 gr per day.

In some embodiments, provided herein is a pharmaceutical composition comprising an oral formulation for the treatment of a neurodegenerative disease. comprising at least 35% Punicic acid-SEDDS comprising full spectrum cannabis oil, CBD, THC, Cinnamon oleoresin alone or mixture thereof with or without mannitol; wherein the ratio between the CBD, THC and Cinnamon oleoresin to the pomegranate or snake gourd oil is between 1:250-1:1 and the total amount of mannitol is between 250 mg to 20 gr per day.

In some embodiments, provided herein is a pharmaceutical composition comprising an oral formulation for the treatment of a neurodegenerative disease. comprising at least 40% Punicic acid-SEDDS comprising full spectrum cannabis oil, CBD, THC, Cinnamon oleoresin alone or mixture thereof with or without mannitol; wherein the ratio between the CBD, THC and Cinnamon oleoresin to the pomegranate or snake gourd oil is between 1:250-1:1 and the total amount of mannitol is between 250 mg to 20 gr per day.

In some embodiments, the ratio between the CBD, THC and Cinnamon oleoresin to the pomegranate or snake gourd oil in the pharmaceutical composition is between 1:120-1:1.

In some embodiments, the ratio between the CBD, THC and Cinnamon oleoresin to the pomegranate or snake gourd oil in the pharmaceutical composition is about 1:120, about 1:100, about 1:80, about 1:60, about 1:50, about 1:30, about 1:20, about 1:15, about 1:12, about 1:10, about 1:8, about 1:5, about 1:2.5, about 1:1.

In some embodiments, the punicic acid-SEDDS is formulated using snake gourd oil.

In some embodiments, the punicic acid-SEDDS is formulated using Pomegranate Seed Oil.

In some embodiments, the pharmaceutical composition comprises a total amount of mannitol is between 250 mg to 18 gr per day. In some embodiments, the pharmaceutical composition comprises a total

amount of mannitol is about 250 mg per day, about 500 mg per day, about 750 mg per day, about 1 g per day, about 3 g per day, about 5 g per day, about 10 g per day, about 12 g per day, about 15 g per day, about 18 g per day.

In some embodiments, the pharmaceutical composition comprises cinnamon oleoresin in an amount of between 1-125 mg.

In some embodiments, the amount of cinnamon oleoresin is between about 1 mg, about 5 mg, about 10 mg, about 20 mg, about 40 mg, about 50 mg, about 70 mg, about 100 mg, about 125 mg.

In some embodiments, the pharmaceutical composition comprises THC in an amount of between 1-125 mg.

In some embodiments, the amount of THC is between about 1 mg, about 5 mg, about 10 mg, about 20 mg, about 40 mg, about 50 mg, about 70 mg, about 100 mg, about 125 mg.

In some embodiments, the pharmaceutical composition comprises CBD in an amount of between 1-125 mg.

In some embodiments, the amount of CBD is between about 1 mg, about 5 mg, about 10 mg, about 20 mg, about 40 mg, about 50 mg, about 70 mg, about 100 mg, about 125 mg.

In some embodiments, the pharmaceutical composition comprises Ethanol extract of Hericium erinaceus in an amount of between 1-125 mg.

In some embodiments, the amount of Ethanol extract of Hericium erinaceus is between about 1 mg, about 5 mg, about 10 mg, about 20 mg, about 40 mg, about 50 mg, about 70 mg, about 100 mg, about 125 mg.

In some embodiments, the pharmaceutical composition further comprises at least one diluent or excipient.

In some embodiments, the pharmaceutical composition wherein the concentrations of the ingredients are Pomegranate Seed Oil or snake gourd oil 20-35% w/w, Tween 80 40-50% w/w, Span 80 15-25% w/w, Ethanol 0-10% w/w, cannabis oil 0-5% w/w, CBD 0-5% w/w, THC 0-5% w/w, Cinnamon oleoresin 0-5% w/w.

In some embodiments, the present invention provides a method for the treatment of a neurodegenerative disease said method comprising administering an effective amount of an oral formulation comprising at least 20% Punicic acid-SEDDS comprising full spectrum cannabis oil, CBD, THC, Cinnamon oleoresin alone or mixture thereof with or without mannitol; wherein the ratio between the CBD, THC and Cinnamon oleoresin to the pomegranate or snake gourd oil is between 1:250-1:1 and total amount of mannitol is between 250 mg to 20 gr per day to a subject in need thereof.

In some embodiments, the present invention provides a method for the treatment of a neurodegenerative disease said method comprising administering an effective amount of an oral formulation comprising at least 25% Punicic acid-SEDDS comprising full spectrum cannabis oil, CBD, THC, Cinnamon oleoresin alone or mixture thereof with or without mannitol; wherein the ratio between the CBD, THC and Cinnamon oleoresin to the pomegranate or snake gourd oil is between 1:250-1:1 and total amount of mannitol is between 250 mg to 20 gr per day to a subject in need thereof.

In some embodiments, the present invention provides a method for the treatment of a neurodegenerative disease said method comprising administering an effective amount of an oral formulation comprising at least 30% Punicic acid-SEDDS comprising full spectrum cannabis oil, CBD, THC, Cinnamon oleoresin alone or mixture thereof with or without mannitol; wherein the ratio between the CBD, THC and Cinnamon oleoresin to the pomegranate or snake gourd oil is between 1:250-1:1 and total amount of mannitol is between 250 mg to 20 gr per day to a subject in need thereof.

In some embodiments, the present invention provides a method for the treatment of a neurodegenerative disease said method comprising administering an effective amount of an oral formulation comprising at least 35% Punicic acid-SEDDS comprising full spectrum cannabis oil, CBD, THC, Cinnamon oleoresin alone or mixture thereof with or without mannitol; wherein the ratio between the CBD, THC and Cinnamon oleoresin to the pomegranate or snake gourd oil is between 1:250-1:1 and total amount of mannitol is between 250 mg to 20 gr per day to a subject in need thereof.

In some embodiments, the present invention provides a method for the treatment of a neurodegenerative disease said method comprising administering an effective amount of an oral formulation comprising at least 40% Punicic acid-SEDDS comprising full spectrum cannabis oil, CBD, THC, Cinnamon oleoresin alone or mixture thereof with or without mannitol; wherein the ratio between the CBD, THC and Cinnamon oleoresin to the pomegranate or snake gourd oil is between 1:250-1:1 and total amount of mannitol is between 250 mg to 20 gr per day to a subject in need thereof.

In some embodiments, the neurogenerative disease is Dementia, Alzheimer's disease, Amyotrophic lateral sclerosis, Friedreich's ataxia, Creutzfeldt-Jakob disease, Pick's disease, amyotrophic lateral sclerosis, neurofibromatosis, brain injury, stroke, multiple sclerosis, loss of memory, multiple infarct dementia Huntington's disease, Lewy body disease, or Parkinson's disease.

In some embodiments, the neurogenerative disease is Dementia.

In some embodiments, the neurogenerative disease is Alzheimer's disease.

In some embodiments, the neurogenerative disease is Amyotrophic lateral sclerosis.

In some embodiments, the neurogenerative disease is Friedreich's ataxia.

In some embodiments, the neurogenerative disease is Creutzfeldt-Jakob disease.

In some embodiments, the neurogenerative disease is Pick's disease.

In some embodiments, the neurogenerative disease is amyotrophic lateral sclerosis.

In some embodiments, the neurogenerative disease is neurofibromatosis.

In some embodiments, the neurogenerative disease is brain injury.

In some embodiments, the neurogenerative disease is stroke.

In some embodiments, the neurogenerative disease is multiple sclerosis.

In some embodiments, the neurogenerative disease is loss of memory.

In some embodiments, the neurogenerative disease is multiple infarct dementia.

In some embodiments, the neurogenerative disease is Huntington's disease.

In some embodiments, the neurogenerative disease is Lewy body disease.

In some embodiments, the neurogenerative disease is Parkinson's disease.

In some embodiments, the pharmaceutical composition as described herein is administered once daily, twice daily, three times per day, 3 times a week, 2 times a week, once a week, once every two weeks, or once a month.

In some embodiments, the pharmaceutical compositions comprising a therapeutically effective amount of a compound of the present disclosure, alone or in combination with one or more pharmaceutically acceptable carriers.

In some embodiments, the pharmaceutical compositions according to the present disclosure are those suitable for enteral, such as oral, transdermal and parenteral administration to a subject, including man, for use in the treatment of neurodegenerative diseases.

In some embodiments, the pharmaceutical composition is suitable for oral administration to a subject for use in the treatment of neurodegenerative diseases, such as Dementia, Alzheimer's disease, Amyotrophic lateral sclerosis, Friedreich's ataxia, Creutzfeldt-Jakob disease, Pick's disease, amyotrophic lateral sclerosis, neurofibromatosis, brain injury, stroke, multiple sclerosis, loss of memory, multiple infarct dementia Huntington's disease, Lewy body disease, or Parkinson's disease.

In some embodiments, the pharmaceutical composition is suitable for oral administration to a subject for use in the treatment of neurodegenerative diseases, such as Dementia, Alzheimer's disease, or Amyotrophic lateral sclerosis, Friedreich's ataxia, Huntington's disease, Lewy body disease, Parkinson's disease.

In one embodiment, the subject is a mammal.

In another embodiment, the subject is a human.

In other embodiments, the subject is a male, female, adult, child, or infant.

Generally, the concentration of the pharmaceutical compositions of the present disclosure in a liquid composition, such as an oral solution, will be from about 0.01-about 50 wt %, preferably from about 0.1-about 10 wt %. The concentration in a semi-solid or a solid composition such as a gel or a powder will be about 0.1-about 5 wt %, preferably about 0.5-about 50 wt %.

The pharmaceutical compositions of the present invention may, for example, be administered in dosage unit formulations containing conventional pharmaceutically acceptable carriers, adjuvants, and vehicles. For example, the pharmaceutical carrier may contain a mixture of mannitol or lactose and microcrystalline cellulose. The mixture may contain additional components such as a lubricating agent, e.g., magnesium stearate and a disintegrating agent such as crospovidone. The carrier mixture may be filled into a gelatin capsule or compressed as a tablet. The pharmaceutical composition may be administered as an oral dosage form, for example.

For oral administration, the pharmaceutical composition may be in the form of, for example, a tablet, capsule, liquid capsule, suspension, or liquid. The pharmaceutical composition is preferably made in the form of a dosage unit containing a particular amount of the active ingredient.

For example, the pharmaceutical composition may be administered in unit dosage form, e.g, comprising 5 to 1000 μg, about 10 to about 750 μg, about 50 to about 500 μg of active ingredient per unit dosage form.

Any pharmaceutical composition contemplated herein can, for example, be delivered orally via any acceptable and suitable oral preparations. Exemplary oral preparations, include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the invention can contain at least one agent selected from sweetening agents, flavoring agents, coloring agents, demulcents, antioxidants, and preserving agents.

Pharmaceutically acceptable carriers, adjuvants, and vehicles that may be used in the pharmaceutical compositions of this invention include, but are not limited to, ion exchangers, alumina, aluminum stearate, lecithin, self-emulsifying drug delivery systems (SEDDS) such as d-alpha-tocopherol polyethyleneglycol 1000 succinate, surfactants used in pharmaceutical dosage forms such as Tweens, polyethoxylated castor oil such as CREMOPHOR® surfactant (BASF), or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2-and 3-hydroxypropyl-cyclodextrins, or other solubilized derivatives may also be advantageously used to enhance delivery of compounds of the formulae described herein.

The pharmaceutically active compounds of this invention can be processed in accordance with conventional methods of pharmacy to produce medicinal agents for administration to subjects, including humans and other mammals. The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such pharmaceutical compositions may also comprise adjuvants, such as wetting, sweetening, flavoring, and perfuming agents.

For therapeutic purposes, the active compounds of this invention are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

In various embodiments, the present disclosure provides pharmaceutical compositions comprising a therapeutically effective amount of the pharmaceutical composition of the disclosure in combination with a therapeutically effective amount of another therapeutic agent.

According to an embodiment, the pharmaceutical compositions may further contain a therapeutically effective amount of said pharmaceutical composition of the disclosure as defined above, either alone or in a combination with another therapeutic agent, e.g., each at an effective therapeutic dose as reported in the art.

### Process of Preparation

An embodiment of the present disclosure relates to preparation of a pharmaceutical composition using an oil comprising at least 20% punicic acid, Tween 80, Span 80 and ethanol, mixed by a magnetic stirrer for 20 min, the mixture is then added to deionized water, vortexed for 30-60 seconds an emulsion.

An embodiment of the present disclosure relates to preparation of a pharmaceutical composition using other self emulsifying systems.

In some embodiments, of the preparation of a pharmaceutical composition the concentrations of the ingredients are 250 mg of an oil comprising at least 20% punicic acid, 350 mg of Tween 80, 155 mg of Span 80 and 50 μl of ethanol, mixed by a magnetic stirrer for 20 min, 10 μl of the mixture is then added to 3 ml of deionized water, vortexed for 30-60 seconds an emulsion.

### Definitions

As used herein, “pharmaceutical composition” means therapeutically effective amounts of a compound of the present invention, together with suitable diluents, preservatives, solubilizers, emulsifiers, adjuvant and/or carriers. Such compositions are liquids or lyophilized or otherwise dried formulations and include diluents of various buffer content (e.g.; Tris-HCL, acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils).

The term “treatment” as used herein refers to the administering of a therapeutic amount of the composition of the present invention which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to reverse the damage caused by the disease, to delay the onset of said progressive stage, to lessen the severity or cure the disease, to improve survival rate or more rapid recovery, or to prevent the disease from occurring or a combination of two or more of the above. in one embodiment, therapeutic treatment and, in another embodiment, prophylactic or preventative measures.

In one embodiment, the goal of treating is to prevent or lessen the targeted pathologic condition or disorder as described hereinabove. Thus, in one embodiment, treating may include directly affecting or curing, suppressing, inhibiting, preventing, reducing the severity of, delaying the onset of, reducing symptoms associated with the disease, disorder or condition, or a combination thereof. Thus, in one embodiment, “treating” refers inter alia to delaying progression, expediting remission, inducing remission, augmenting remission, speeding recovery, increasing efficacy of or decreasing resistance to alternative therapeutics, or a combination thereof. In one embodiment, “preventing” refers, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging subject survival, or a combination thereof.

The “effective amount” for purposes disclosed herein is determined by such considerations as may be known in the art. The amount must be effective to achieve the desired therapeutic effect as described above, depending, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile within the body, a variety of pharmacological parameters such as half life in the body, on undesired side effects, if any, on factors such as age and gender, etc.

The pharmaceutical compositions of the invention may comprise additionally any other suitable substances such as other therapeutically useful substances, diagnostically useful substances, pharmaceutically acceptable carriers or the like.

The phrase “pharmaceutically acceptable” is employed herein to refer to those compounds, materials, compositions, combinations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any integer or step or group of integers and steps.

The present invention further comprises combinations of the compositions of the present invention and, optionally, one or more additional agents in kit form, e.g., where they are packaged together or placed in separate packages to be sold together as a kit, or where they are packaged to be formulated together.

If desired, the pharmaceutical composition(s) are provided together with instructions for administering the pharmaceutical composition(s) to a subject having or at risk of developing a neurodegenerative disease. The instructions will generally include information about the use of the composition for the treatment or prevention of a neurodegenerative disease. In other embodiments, the instructions include at least one of the following: description of the therapeutic agent; dosage schedule and administration for treatment or prevention of a neurodegenerative disease or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The following Examples are representative of techniques employed by the inventors in carrying out aspects of the present invention. It should be appreciated that while these techniques are exemplary embodiments for the practice of the invention, those of skill in the art, in light of the present disclosure, will recognize that numerous modifications can be made without departing from the spirit and intended scope of the invention.

### EXAMPLES

### Example 1

### Preparation of Pomegranate/Snake Gourd Oil Self-Emulsifying Drug Delivery System (SEDDS)

250 mg of Pomegranate or snake gourd oil or any other oil comprising at least 20% punicic acid, 350 mg of Tween 80, 155 mg of Span 80 and 50 μl of ethanol are mixed by a magnetic stirrer for 20 min. 10 μl of the mixture is then added to 3 ml of deionized water. After vortexing for 30-60 seconds, an emulsion with nano-sized droplets is obtained.

Components concentrations at the SEDDS: 31.46% oil 44% Tween 80 19.5% Span 80 4.97% Ethanol The O/W nanoemulsion droplets size expected is: 224 nm Z Average, peak 1: 181 nm, 100%; Pdi: 0.389. according to Gabizon et al., (EP2844265A4). The following combinations of Punicic acid-SEDDS formulations with one or more of the active compounds can be prepared. In all formulations, the total oil content is 250 mg:
1. 1-125 mg cinnamon oleoresin
2. 1-125 mg THC
3. 1-125 mg CBD
4. 1-125 mg Ethanol extract of Hericium erinaceus
5. 250 mg-20gr mannitol

Table 1 shows components of the SEDDS according to some embodiments

TABLE 1

| | 1 | 2 | 3 | 4 | 5 | 6 | 7 | 8 | 9 | 10 |
|---|---|---|---|---|---|---|---|---|---|---|
| mannitol | V | V | V | V | V | V | V | V | V | V |
| pomegranate oil | V | V | V | V | V | | | | | |
| snake gourd oil | | | | | | V | V | V | V | V |
| selected full | | V | | | | | V | | | |
| spectrum cannabis | | | | | | | | | | |
| THCisolate | | | V | | | | | V | | |
| (Ultra low THC) | | | | | | | | | | |
| cinnamon oil | | | | V | | | | | V | |
| Hericium erinaceus | | | | | V | | | | | V |
| hericenones\erinacines | | | | | | | | | | |

### Example 2

### Formulations

SEDDS formulations with snake gourd oil and Pomegranate Seed Oil (PSO) with a combination of additional lipophilic compounds from plant source.

Description of formulation: Final SEDDS formulations with additional lipophilic compounds from plant sources were prepared and evaluated at 40° C. for 12 weeks.

Formulations' design and characterization-Four different SEDDS formulations were prepared and evaluated:

Formulations I and II: SEDDS-PSO formulation including and excluding ethanol.

Prior to formulation's preparation, PSO was mixed with pomegranate fruit extract and filtered.

After that, the Formulations were prepared including additional oil components as described in Tables 2 and 3.

The formulations were filled into a 1.5 ml amber vial with no headspace to mimic as much as possible capsules environment and to avoid unwanted chemical reactions.

Formulations III and IV: SEDDS-snake gourd oil formulation including and excluding ethanol.

Formulations comprising snake gourd oil, which showed no properties sensitive to environmental conditions, were mixed with additional oil components as described in Tables 4 and 5.

These formulations were filled in 20 ml glass vials (with a headspace of about 50%).

All formulations were kept at 40° C. in dark conditions. Quantitative formulations and characterization are described in tables 2-5.

Table 2 (Formulation I) shows the quantitative formulations and characterization for SEDDS-PSO formulations with ethanol.

TABLE 2

| | Formulation # | | | | | | | |
|---|---|---|---|---|---|---|---|---|
| | 1 | | 2 | | 3 | | 4 | |
| | Ingredient | % (w/w) | Ingredient | % (w/w) | Ingredient | % (w/w) | Ingredient | % (w/w) |
| Formulation | PSO | 30 | PSO | 27.36 | PSO | 27.7 | PSO | 26.47 |
| | Tween 80 | 45 | Cannabis extract | 2.64 | Cinnamon oil | 2.3 | Cannabis extract | 2.53 |
| | Span 80 | 20 | Tween 80 | 45 | Tween 80 | 45 | Cinnamon oil | 2.2 |
| | Ethanol | 5 | Span 80 | 20 | Span 80 | 20 | Tween 80 | 45 |
| | | | Ethanol | 5 | Ethanol | 5 | Span 80 | 20 |
| | | | | | | | Ethanol | 5 |

| | | | | |
|---|---|---|---|---|
| Appearance | Slightly opaque | Slightly opaque | Slightly opaque | Slightly opaque |
| Package | Full amber | Full amber | Full amber | Full amber |
| | vial (1.5 ml) | vial (1.5 ml) | vial (1.5 ml) | vial (1.5 ml) |
| Stability Results | Stable | Stable | Stable | Stable |
| (4 weeks) | | | | |
| Self-emulsifying | v | v | v | v |
| CBD conc. (%) within | NA | 2.32 | NA | 2.22 |
| the extract T ‘0’ | | | | |
| CBD conc. (%) within | NA | 2.32 | NA | 2.22 |
| the extract 1 month | | | | |
| Particle size | NA | NA | NA | NA |

Table 3: (Formulation II) shows: the quantitative formulations and characterization for ethanol-free SEDDS-PSO formulations:

TABLE 3

| | Formulation # | | | | | | | |
|---|---|---|---|---|---|---|---|---|
| | 5 | | 6 | | 7 | | 8 | |
| | Ingredient | % (w/w) | Ingredient | % (w/w) | Ingredient | % (w/w) | Ingredient | % (w/w) |
| Formulation | PSO | 30 | PSO | 27.3 | PSO | 27.7 | PSO | 26.6 |
| | Tween 80 | 45 | CBD extract | 2.64 | Cinnamon oil | 2.3 | CBD extract | 2.53 |
| | Span 20 | 5 | Tween 80 | 45 | Tween 80 | 45 | Cinnamon oil | 2.2 |
| | | | Span 20 | 5 | Span 20 | 5 | Tween 80 | 45 |
| | | | | | | | Span 20 | 5 |

| | | | | |
|---|---|---|---|---|
| Appearance | Slightly opaque | Slightly opaque | Slightly opaque | Slightly opaque |
| Package | Full amber | Full amber | Full amber | Full amber |
| | vial (1.5 ml) | vial (1.5 ml) | vial (1.5 ml) | vial (1.5 ml) |
| Stability Results | Stable | Stable | Stable | Stable |
| (4 weeks) | | | | |
| Self-emulsifying | v | v | v | v |
| Particle size | NA | NA | NA | NA |

Table 4: (Formulation III) shows: The quantitative formulations and characterization for SEDDS-snake gourd oil formulations with ethanol:

TABLE 4

| | Formulation # | | | | | | | | | |
|---|---|---|---|---|---|---|---|---|---|---|
| | 9 | | 10 | | 11 | | 12 | | 13 | |
| | Ingredient | % (w/w) | Ingredient | % (w/w) | Ingredient | % (w/w) | Ingredient | % (w/w) | Ingredient | % (w/w) |
| Formulation | Snake | 30 | Snake | 27.36 | Snake | 27.7 | Snake | 25.27 | Snake | 27.10 |
| | Gourd oil | | Gourd oil | | Gourd oil | | Gourd oil | | Gourd oil | |
| | Tween 80 | 45 | Cannabis | 2.64 | Cinnamon | 2.3 | Cannabis | 2.53 | Cannabis | 2.64 |
| | Span 80 | 20 | extract | | oil | | extract | | extract | |
| | Ethanol | 5 | Tween 80 | 45 | Tween 80 | 45 | Cinnamon | 2.2 | THC | 0.264 |
| | | | Span 80 | 20 | Span 80 | 20 | oil | | | |
| | | | Ethanol | 5 | Ethanol | 5 | Tween 80 | 45 | Tween 80 | 45 |
| | | | | | | | Span 80 | 20 | Span 80 | 20 |
| | | | | | | | Ethanol | 5 | Ethanol | 5 |

| | | | | | |
|---|---|---|---|---|---|
| Appearance | Slightly opaque | Slighty opaque | Slighty opaque | Slightly opaque | Slightly opaque |
| Stability | Stable | Stable | Stable | Stable | Stable |
| Results | | | | | |
| (4 weeks) | | | | | |
| Self- | v | v | v | v | v |
| emulsifying | | | | | |
| CBD conc. (%) | NA | 2.32 | NA | 2.22 | 2.23 |
| within the | | | | | |
| extract T ‘0’ | | | | | |
| CBD conc. (%) | NA | 2.31 | NA | 2.21 | 2.31 |
| within the | | | | | |
| extract | | | | | |
| 1 month | | | | | |
| Particle size | NA | NA | NA | NA | NA |

Table 5: (Formulation IV) show: The quantitative formulations and characterization for ethanol-free SEDDS snake gourd oil formulations:

TABLE 5

| | Formulation # | | | | | | | |
|---|---|---|---|---|---|---|---|---|
| | 14 | | 15 | | 16 | | 17 | |
| | Ingredient | % (w/w) | Ingredient | % (w/w) | Ingredient | % (w/w) | Ingredient | % (w/w) |
| Formulation | Snake | 30 | Snake | 27.7 | Snake | 27.36 | Snake | 25.27 |
| | Gourd oil | | Gourd oil | | Gourd oil | | Gourd oil | |
| | Tween 80 | 45 | Cinnamon | 2.3 | Cannabis | 2.64 | Cannabis | 2.53 |
| | Span 20 | 5 | oil | | extract | | extract | |
| | | | Tween 80 | 45 | Tween 80 | 45 | Cinnamon | 2.2 |
| | | | Span 20 | 25 | Span 20 | 25 | oil | |
| | | | | | | | Tween 80 | 45 |
| | | | | | | | Span 20 | 25 |

| | | | | |
|---|---|---|---|---|
| Appearance | Slightly opaque | Slightly opaque | Slightly opaque | Slightly opaque |
| Stability Results | Stable | Stable | Stable | Stable |
| Self-emulsifying | v | v | v | v |
| Particle size | NA | NA | NA | NA |

All ingredients were dissolved completely and formed homogenous solutions. Moreover, all formulations demonstrated self-emulsifying ability and remained stable for four weeks.

### Conclusions

After four weeks, all Formulations remained stable. No observed viscous ring on the SEDDS-PSO formulations' surface appeared.

### Example 3

### In-Vitro Assays

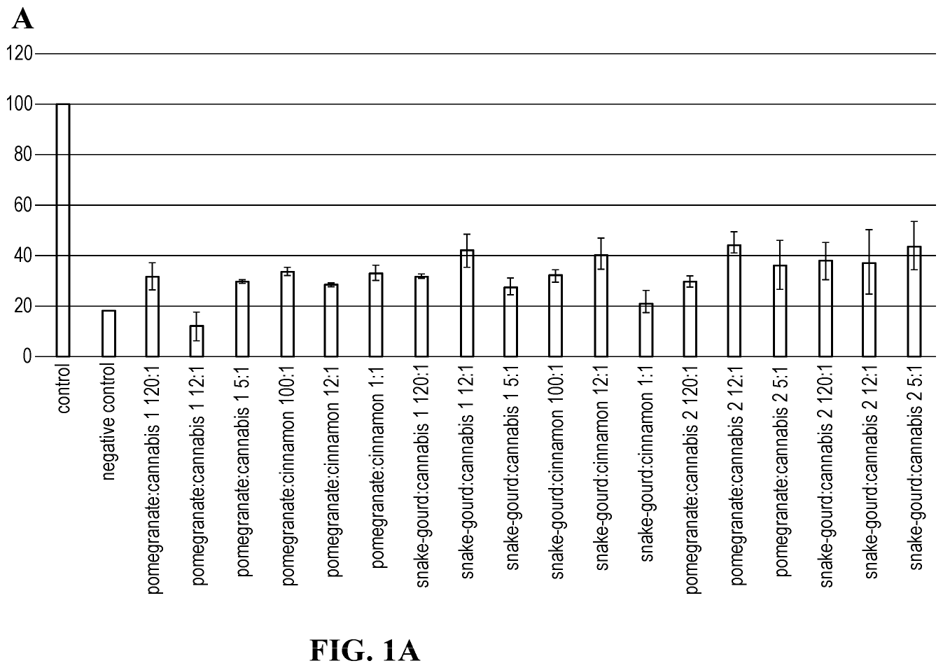

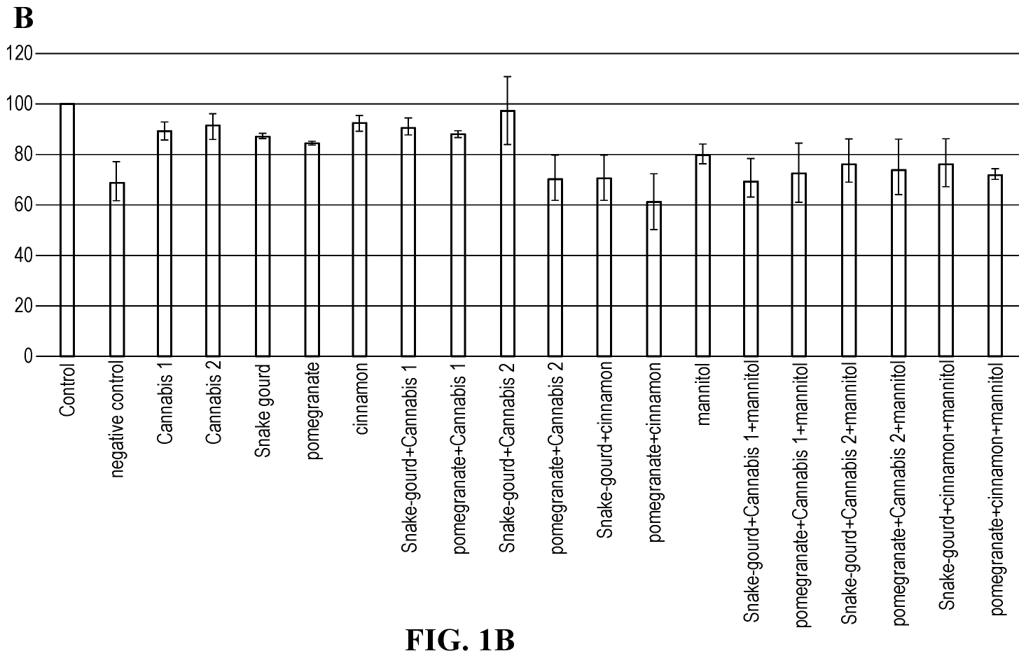

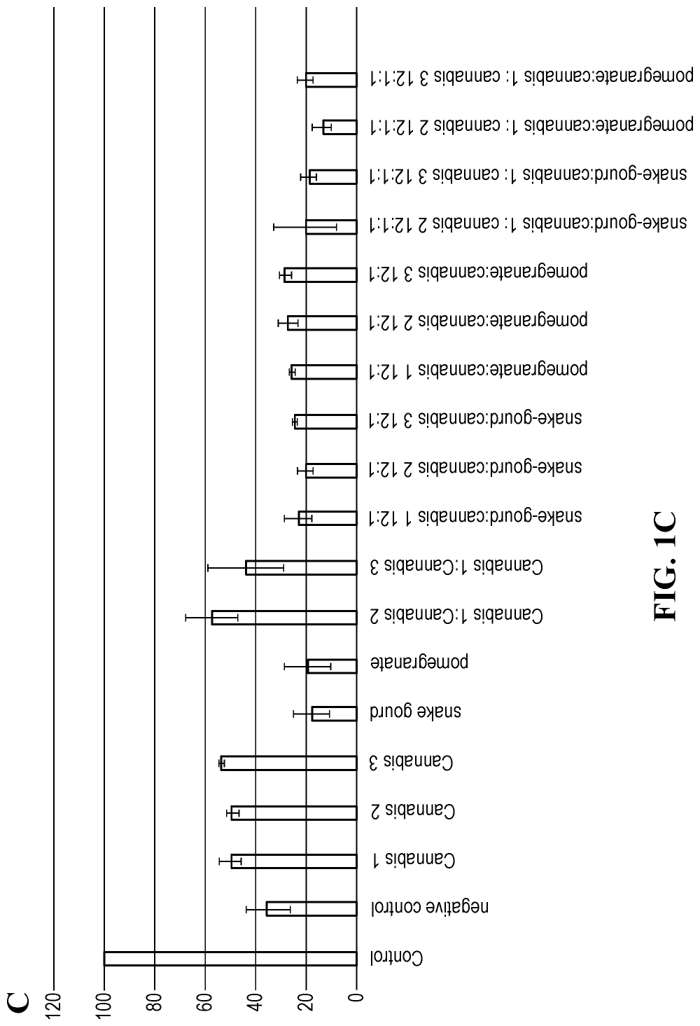

The neuroprotective effect of different oil combinations was tested on human neuroblastoma cell line SHSY-5Y neurons. A variety of combinations according to the formulation preparation were screened (FIG. 1A). FIG. 1A shows the ratio screen.

The ratio between snake-gourd or pomegranate to cannabis or cinnamon was aligned with the formulation development. Combining the results obtained from the two screening assays, the ratio of 12:1 (snake-gourd or pomegranate:cannabis or cinnamon respectively) was found most effective.

Three cannabis extracts were used in the combination screen (FIGS. 1B and 1C). Pomegranate or snake-gourd to cannabis or cinnamon at 12:1 ratio in different combination screens is shown in FIG. 1B.

Pomegranate or snake-gourd to cannabis or cinnamon at 12:1 ratios in different combination screens without mannitol is shown in FIG. 1C.

In the experiments in FIGS. 1b and FIG. 1c SHSY-5Y neurons were treated with oil combination for 24 h, prior to 24 h of 300 μM H₂O₂ administration. Relative viability of the cells was determined by MTT method. Data in the figures are presented as mean±S.E. n=3.

The figures illustrate that the Pomegranate or snake-gourd to cannabis or cinnamon 12:1 ratio was approximately 70% effective in providing neuroprotection in SHSY-5Y neurons with mannitol and approximately 20% effective in providing neuroprotection in SHSY-5Y neurons without mannitol.

### Example 4

### In-Vivo Assays

The in vivo Parkinson Drosophila model is tested as described by Shaltiel-Karyo (2013). The advantage of this model is that the generation time is relatively short (about 10 days) compared to other animal models such as mice.

An in vivo mouse model—cognitive function restoration in old mice—is tested as described by Bilkei-Gorzo et al. (2017). This model uses different Male C57BL/6J mice aged 2 months (young), 12 months (mature) and 18 months (old).

### Behavioral Testing

The novel object location recognition test was performed in a sound-isolated, dimly illuminated room in an open-field box (44 cm×44 cm). The floor was covered with sawdust (1 cm deep, used and saturated with the odor of the animals).

The habituation period consisted of a daily 5-min period of free exploration in the arena containing three objects (plastic balls, 15 mm in diameter) for 3 day. On the test day, the animals were allowed to explore three identical objects (Lego pieces with different colors, roughly 2×2 cm) placed into the area in a fixed location for 6 min, and the time spent on inspection of the individual objects was recorded (Noldus Ethovision XT). Thirty minutes later, the animals were placed back into the box, where one object was placed into a new location. The animals were left to explore for an additional 3 min. The time spent with investigations was recorded, and the preference ratio for the moved object was calculated as follows: preference=Ta/(Ta+Tb+Tc)×100; T, time spent with investigation; Ta, the object that is moved in the second trial; Tb and Tc, the objects that remained in their original positions. Novelty preference was calculated as follows: (Pt2−Pt1)/Pt1×100; P, the preference of the mouse; t1, trial one; t2, trial two. Long-term memory was tested using a modified form of the partner recognition test 42 days after the minipump implantation. The test was performed in the same arenas and after the same habituation as described for the novel object location recognition test. In the first trial, the arenas held both a metal grid cage only containing a mouse (of the same age and sex as the test animal but from a different cage) and one other object (of a similar size and form as the metal grid cage) in the opposing corner, placed 6-7 cm from the walls. The location and activity of the test mouse were recorded and analyzed by the Etho Vision tracking system (Noldus) for 15 min. In the next session, after 24 h, the object was replaced with another grid cage containing a new partner, and the activity of the test mouse was recorded again for 5 min. Recognition of the previously seen partner was defined by a novelty preference, i.e., a significantly longer period spent investigating the new partner in the second trial. Novelty preference was calculated as Ta/(Ta+Tb)×100; Ta is the time spent with the novel partner; Tb is the time spent with the previous partner.

Spatial learning and memory were assessed in the MWM task (described in Albayram Proc. Natl Acad. Science USA 108, 112256-11261 (2011); Barnes, Nature 388, 272-275 (1997); Sanchez-Mejia Nat. Neurosci. 11, 1311-1318 (2008)). In the acquisition phase of the MWM test, the animals were tested for four consecutive sessions daily over 5 days. The hidden platform remained at a fixed spatial location for the entire acquisition period. The mice started from the same position at days 1 and 2 and from variable positions in the following trials. Long-term spatial memory was assessed at day 6, when the platform was removed and the time spent in the platform-associated quadrant was measured. We assessed the flexibility of spatial memory by placing the platform in a new location (reversal phase) between days 7 and 9. Animals that did not move (floaters) or just circled in close vicinity to the wall (wall-hangers) were excluded from the analysis and further tests. The criteria were pre-established. The investigator was blinded to genotype or treatment, but the difference between the age groups was clearly visible. Recording and analysis of behavior were carried out by automated systems (Videomot, TSE-Systems).

The effect of pomegranate-CBD based formulation with a 12:1 pomegranate to CBD ratio on cognitive decline was further evaluated in C57BL6 mice. The cognitive decline of the mice was achieved using MK-801 and the cognitive ability of the mice were tested using the Y-maze Test. The experiment included three groups of mice, neutral control (N=12), MK-801 (N=12), MK-801 Pomegranate-CBD treated (N=12).

Results indicate that while neutral control mice entered the novel arm of the Y-maze in 41.7% of entries, an amount which is higher than random (i.e. 33% of the three maze arms), the MK-801 group entered the novel arm of the Y-maze in 31.8% of entries in the novel arm, which is closer to (and even lower than) random (i.e. 33%). This demonstrates that the MK-801 mice experienced a cognitive decline which caused them to enter the novel arm only as much as they did to any of the other arms. This contrasts with the neutral control that entered the novel arm more often (41.7%).

The 12:1 pomegranate-CBD based formulation administered to the MK-801 pomegranate-CBD treated group completely reversed the effect of MK-801 in a statistically significant manner, resulting in 39.3% of entries in the novel arm. MK-801 pomegranate-CBD treated group entries in the novel arm were statistically insignificant compared with the neutral control mice group.

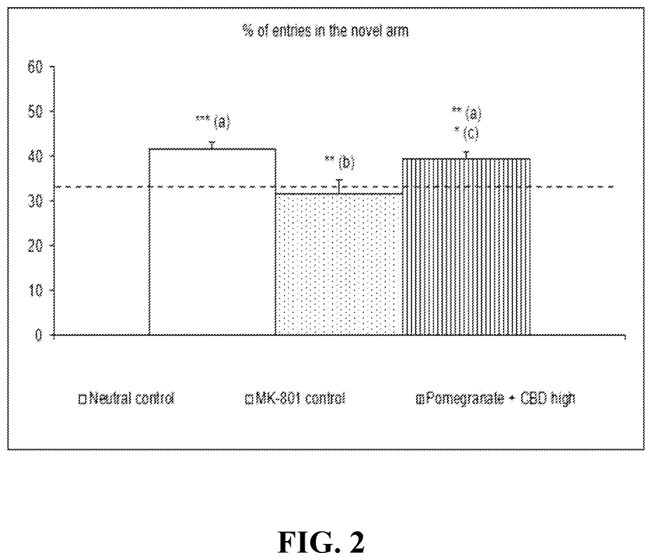

Results are presented in FIG. 2. The results demonstrate the in vivo cognitive effect of the pomegranate-CBD combination at a 12:1 ratio.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A pharmaceutical composition comprising at least 20% Punicic acid-Self-Emulsifying Drug Delivery System (SEDDS) comprising any one of full spectrum cannabis oil, CBD, THC, Cinnamon oleoresin, or any combination thereof.

2. The pharmaceutical composition of claim 1 wherein a ratio between the full spectrum cannabis oil, CBD, THC, and Cinnamon oleoresin to pomegranate or snake gourd oil is between 1:250-1:1.

3. The pharmaceutical composition of claim 1 wherein the pharmaceutical composition is an oral formulation.

4. The pharmaceutical composition of claim 1, further comprising mannitol.

5. The pharmaceutical composition of claim 1, wherein the ratio between the CBD, THC and Cinnamon oleoresin to pomegranate or snake gourd oil is between 1:120-1:1.

6. The pharmaceutical composition of claim 5, wherein the ratio between the CBD, THC and Cinnamon oleoresin to the pomegranate or snake gourd oil is between 1:12.

7. The pharmaceutical composition of claim 1, wherein the punicic acid-SEDDS is formulated using snake gourd oil.

8. The pharmaceutical composition of claim 1, wherein the punicic acid-SEDDS is formulated using Pomegranate Seed Oil.

9. The pharmaceutical composition of claim 1, wherein the total amount of mannitol is between 250 mg to 18 gr per day.

10. The pharmaceutical composition of claim 1, free of mannitol.

11. The pharmaceutical composition according to claim 1, further comprising at least one diluent or excipient.

12. The pharmaceutical composition of claim 11, wherein the concentrations of the ingredients are Pomegranate Seed Oil or snake gourd oil 20-35% w/w, Tween 80 40-50% w/w, Span 80 15-25% w/w, Ethanol 0-10% w/w, cannabis 0-5% w/w oil, CBD 0-5% w/w, THC, Cinnamon oleoresin 0-5% w/w.

13. The pharmaceutical composition according to claim 1, wherein the composition is substantially stable after four weeks.

14. The pharmaceutical composition of claim 1 wherein the Punicic acid-SEDDS is prepared using 250 mg of an oil comprising at least 20% punicic acid, 350 mg of Tween 80, 155 mg of Span 80 and 50 μl of ethanol, mixed by a magnetic stirrer for 20 min, 10 μl of the mixture is then added to 3ml of deionized water, vortexed for 30-60 seconds per emulsion.

15. The pharmaceutical composition of claim 1 for use in the treatment of a neurodegenerative disease.

16. A method for the treatment of a neurodegenerative disease, said method comprising administering an effective amount of an oral formulation comprising at least 20% Punicic acid-SEDDS comprising any one of full spectrum cannabis oil, CBD, THC, Cinnamon oleoresin, or any combination thereof to a patient in need thereof.

17. The method of claim 16, wherein a ratio between the full spectrum cannabis oil, CBD, THC, and Cinnamon oleoresin to pomegranate or snake gourd oil is between 1:250-1:1.

18. The method of claim 16, wherein the neurogenerative disease is Dementia, Alzheimer's disease, Amyotrophic lateral sclerosis, Friedreich's ataxia, Huntington's disease, Lewy body disease, or Parkinson's disease.

19. The method of claim 18, wherein the composition is administered once daily, twice daily or three times per day.

20. A method for preparing the pharmaceutical composition of claim 1, comprising the following steps:
a. Mixing 250 mg of an oil comprising at least 20% punicic acid, 350 mg of Tween 80, 155 mg of Span 80 and 50 μl of ethanol using a magnetic stirrer for 20 minutes.
b. Adding 10 μl of the mixture obtained in the preceding step to 3 ml of deionized water and vortexing for 30-60 seconds.
